(19) United States
(12) Patent Application Publication
(10) Pub. No.: US 2008/0284429 A1
(43) Pub. Date: Nov. 20, 2008

(54) Situ Hyperpolarization of Imaging Agents

(75) Inventors: Charles M. Marcus, Cambridge, MA (US); Jacob W. Aptekar, Cambridge, MA (US); Alexander C. Johnson, Cambridge, MA (US); Ronald L. Walsworth, Cambridge, MA (US)

Correspondence Address: CHOATE, HALL & STEWART LLP, TWO INTERNATIONAL PLACE, BOSTON, MA, 02110, US

(73) Assignee: The President and Fellows of Harvard College, Cambridge, MA (US)

(21) Appl. No.: 12/096,678

(22) PCT Filed: Dec. 11, 2006
(86) PCT No.: PCT/US06/47205

§ 371 Date: Jun. 9, 2008

Related U.S. Application Data
(60) Provisional application No. 60/748,857, filed on Dec. 10, 2005.
(60) Provisional application No. 60/758,245, filed on Jan. 11, 2006.
(60) Provisional application No. 60/783,202, filed on Mar. 16, 2006.

(51) Int. Cl. A61K 49/16; A61K 49/06; G01R 33/56

(52) U.S. Cl. 324/307; 424/9.3; 424/9.37; 424/9.34; 977/734

(57) ABSTRACT

The present invention generally relates to compositions, systems and methods for inducing nuclear hyperpolarization in imaging agents after they have been introduced into a subject.

### PRIORITY INFORMATION

This application claims priority to U.S. Ser. No. 60/748,857 filed Dec. 10, 2005. This application also claims priority to U.S. Ser. No. 60/758,245 filed Jan. 11, 2006. This application also claims priority to U.S. Ser. No. 60/783,202 filed Mar. 16, 2006. The entire contents of these applications are hereby incorporated by reference.

### BACKGROUND OF THE INVENTION

Magnetic resonance imaging (MRI) systems generally provide for diagnostic imaging of regions within a subject by detecting the precession of the magnetic moments of atomic nuclei in an applied external magnetic field. Spatial selectivity, allowing imaging, is achieved by matching the frequency of an applied radio-frequency (rf) oscillating field to the precession frequency of the nuclei in a quasi-static field. By introducing controlled gradients in the quasi-static applied field, specific slices of the subject can be selectively brought into resonance. By a variety of methods of controlling these gradients in multiple directions, as well as controlling the pulsed application of the rf resonant fields, three-dimensional images representing various properties of the nuclear precession can be detected, giving information about the density of nuclei, their environment, and their relaxation processes. By appropriate choice of the magnitude of the applied quasi-static field and the rf frequency, different nuclei can be imaged.

Typically, in medical applications of MRI, it is the nuclei of hydrogen atoms, i.e., protons, that are imaged. This is, of course, not the only possibility. Information about the environment surrounding the nuclei of interest can be obtained by monitoring the relaxation process whereby the precessional motion of the nuclei is damped, either by the relaxation of the nuclear moment orientation returning to alignment with the quasi-static field following a tipping pulse (on a time scale T1), or by the dephasing of the precession due to environmental effects that cause more or less rapid precession, relative to the applied rf frequency (on a time scale T2). Conventional MRI contrast agents, such as those based on gadolinium compounds, operate by locally altering the T1 or T2 relaxation processes of protons. Typically, this relies on the magnetic properties of the contrast agent, which alters the local magnetic environment of protons. In this case, when images display either of these relaxation times as a function of position in the subject, the location of the contrast agent shows up in the image, providing diagnostic information. Contrast enhancement has also been achieved by utilizing the Overhauser effect, in which an electron transition in a paramagnetic contrast agent is coupled to the nuclear spin system of the endogenous imaging nuclei (e.g., protons). This so-called Overhauser-enhanced magnetic resonance imaging (OMRI) technique increases the polarization of the imaged nuclei and thereby amplifies the acquired signal.

An alternative approach to MRI imaging is to introduce into the subject an imaging agent, the nuclei of which themselves are imaged by the techniques described above. That is, rather than affecting the local environment of endogenous protons in the body and thereby providing contrast in a proton image, the exogenous imaging agent is itself imaged. Such imaging agents include atomic and molecular substances that have non-zero nuclear spin such as ³He, ¹²⁹Xe, ³¹P, ²⁹Si, ¹³C and others (e.g., see U.S. Patent Application Publication 2004/0171928). The nuclei in these substances may be polarized ex vivo by various methods (including optically or using sizable applied magnetic fields at room or low temperature) which orient a significant fraction of the nuclei in the agent. The hyperpolarized substance is then introduced into the body. Once in the body, a strong imaging signal is obtained due to the high degree of polarization of the imaging agent. Also there is only a small background signal from the body, as the imaging agent has a resonant frequency that does not excite protons in the body. For example, U.S. Pat. No. 5,545,396 discloses the use of hyperpolarized noble gases for MRI.

Many proposed imaging agents for hyperpolarized MRI have short spin-lattice relaxation (T1) times, requiring that the material be quickly transferred from the hyperpolarizing apparatus to the body, and imaged very soon after introduction into the body, often on the time scale of tens of seconds. For a number of applications, it is desirable to use an imaging agent with longer T1 times. Compared to gases, solid or liquid materials usually lose their hyperpolarization rapidly. Hyperpolarized substances are, therefore, typically used as gases. For example, U.S. Pat. No. 6,453,188 discloses a method of providing magnetic resonance imaging using a hyperpolarized gas that claims to provide a T1 time of several minutes. Protecting even the hyperpolarized gas from losing its magnetic orientation, however, is also difficult in certain applications. For example, U.S. Patent Application Publication No. 2003/0009126 discloses the use of a specialized container for collecting and transporting ³He and ¹²⁹Xe gas while minimizing contact induced spin relaxation. U.S. Pat. No. 6,488,910 discloses providing ¹²⁹Xe gas or ³He gas in microbubbles that are then introduced into the body. The gas is provided in the microbubbles for the purpose of increasing the T1 time of the gas. The spin-lattice relaxation time of such gas, however, is still limited.

There is a need, therefore, for imaging agents that provide greater flexibility in designing relaxation times during nuclear magnetic resonance imaging. In particular, there is a need for hyperpolarizable imaging agents with longer T1 times than those already available. Additionally or alternatively, there is a need for imaging agents and accompanying methods that enable imaging agents to be hyperpolarized in situ, i.e., after they have been introduced into a subject.

### SUMMARY OF THE INVENTION

The present invention generally relates to compositions, systems and methods for inducing nuclear hyperpolarization in imaging agents after they have been introduced into a subject (i.e., in situ hyperpolarization). The imaging agents are solid-state materials that include both non-zero spin nuclei and zero-spin nuclei. In one aspect, the solid imaging agent also includes unpaired electrons and the non-zero spin nuclei are hyperpolarized by placing the subject within an applied magnetic field and irradiating the subject with radiation that penetrates the subject and excites electron spin transitions in the unpaired electrons. In another aspect, the unpaired electrons are not present at the time of administration but are generated optically using a second source of radiation that also penetrates the subject.

### BRIEF DESCRIPTION OF THE DRAWING

The invention is described with reference to the several figures of the drawing, in which:

FIG. 1 is a graph showing measurements of the T1 time for various silicon materials, including micron-scale powders. As shown, T1 times of greater than 1 hour can be achieved in a variety of materials.

FIG. 2 is a schematic illustration of one embodiment of an imaging agent which includes a suspension of particles 10 (optionally modified to include targeting agents). The particles are administered to a subject by injection and can be hyperpolarized in situ after they reach their target site. Within each particle, the concentration of host material atoms 20 that carry a non-zero nuclear spin 30 and the concentration of impurity atoms that provide unpaired electrons 40 can be controlled when the material is synthesized.

### DESCRIPTION OF CERTAIN EMBODIMENTS OF THE INVENTION

This application refers to published documents including patents, patent applications and articles. Each of these published documents is hereby incorporated by reference.

### Introduction

The present invention generally relates to compositions, systems and methods for inducing nuclear hyperpolarization in imaging agents after they have been introduced into a subject. Throughout this application, the shorthand reference “in situ hyperpolarization” will be used to capture this concept. In contrast, prior art methods that involve hyperpolarizing imaging agents before they are introduced into a subject, are given the shorthand reference “ex vivo hyperpolarization.” As discussed in the background section, ex vivo hyperpolarization of imaging agents suffers from a number of limitations that result from nuclear spin relaxation. Indeed, as a consequence of nuclear spin relaxation, the time available between administration of the hyperpolarized agent and signal acquisition is limited by the T1 time. The development of imaging agents with longer T1 times provides a partial solution to this problem by lengthening the potential window between administration and acquisition. However, the ability to hyperpolarize imaging agents in situ removes the limitation entirely. Thus, using in situ hyperpolarization, unpolarized imaging agents can be introduced into a subject and then hyperpolarized hours, days, weeks or even years later. This is particularly useful for imaging agents that cannot reach desired areas of the subject (e.g., a tumor) within the T1 time. In addition, the user can reduce or even remove the delay between hyperpolarization and acquisition thereby enhancing the acquired signal strength. In situ hyperpolarization also opens up the possibility of repeating the hyperpolarization and acquisition cycle multiple times. In certain embodiments this can be used to further enhance signal strength by signal averaging. In other embodiments this can be used to monitor the spatial progress of the imaging agent over time.

### Imaging Agents

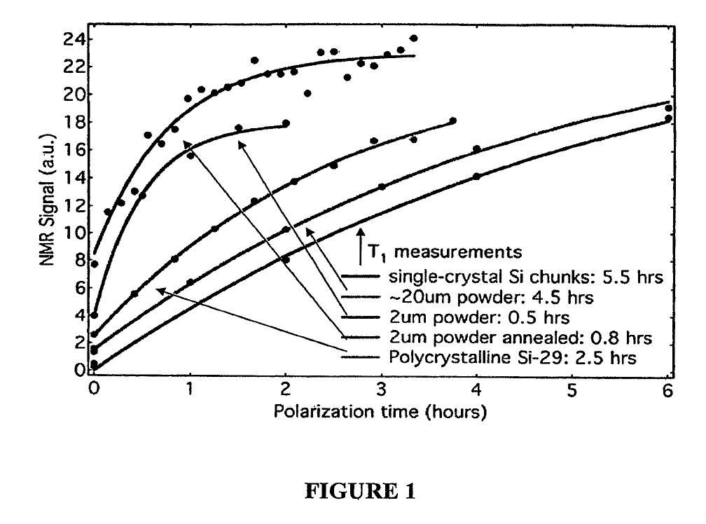

The in situ hyperpolarization methods of the present invention are performed with solid-state imaging agents. Although liquids and solids typically have short relaxation (T1) times, we have discovered that certain solid materials with long T1 times can be manufactured and that these materials can be used as hyperpolarizable imaging agents. For example, FIG. 1 shows measurements of the T1 time for various silicon materials, including micron-scale powders. As shown, T1 times of greater than one hour can be achieved in a variety of materials. It is to be understood that, while the inventive methods enable the preparation and use of materials with long T1 times, the present invention is not limited to such materials. Thus in general, inventive materials may have T1 times that are shorter than one minute, longer than one minute, longer than ten minutes, longer than thirty minutes, longer than one hour, longer than two hours, or even longer than four hours.

The inventive solid materials include both non-zero spin nuclei and zero-spin nuclei (e.g., without limitation, 28Si, 12C, etc.). In certain embodiments, the non-zero spin nuclei are spin-½ nuclei (e.g., without limitation, 129Xe, 29Si, 31P, 19F, 15N, 13C, 3He, etc.). However, other non-zero spin nuclei may be used, e.g., without limitation, 10B which is a spin-3 nucleus and/or 11B which is a spin-3/2 nucleus. The solid material can include a mixture of different non-zero spin nuclei. The solid material can also include a mixture of different zero-spin nuclei.

It is to be understood that the relative concentrations of zero-spin and non-zero spin nuclei within the solid material can be tailored by the user. In one embodiment, the concentration of zero-spin nuclei is greater than the concentration of non-zero spin nuclei. For example, the concentration of non-zero spin nuclei can be less than 50%, less than 40%, less than 30%, less than 20%, less than 10%, less than 5%, less than 1% or even less than 0.1% of the total concentration of nuclei in the solid material. In another embodiment, the concentration of non-zero spin nuclei is greater than the concentration of zero-spin nuclei. For example, the concentration of zero spin nuclei can be less than 50%, less than 40%, less than 30%, less than 20%, less than 10%, less than 5%, less than 1% or even less than 0.1% of the total concentration of nuclei in the solid material. In certain embodiments, different isotopes of a particular element can be present at about natural abundance levels. Alternatively, the solid material may be enriched or depleted for a particular isotope. Methods for preparing such materials have been described, e.g., Ager et al., J. Electrochem. Soc. 152:G488, 2005 describes methods for preparing isotopically enriched silicon.

In one aspect, the solid material may include a mixture of an atomic substance that has no nuclear spin and an atomic substance that has a non-zero nuclear spin. For example, 28Si and 12C have no nuclear spin while 129Xe, 29Si, 31P, 19F, 15N, 13C and 3He have spin-½ nuclei. In one embodiment, the material includes silicon nuclei with a natural abundance mixture of isotopes 28Si (zero-spin, about 92.2%), 29Si (spin-½, about 4.7%) and 30Si (zero-spin, about 3.1%). In another embodiment, the level of 29Si is higher than its natural abundance level, e.g., higher than about 4.7%, 5%, 7%, 10%, 20%, 30%, 40% or even 50%. In yet another embodiment, the level of 29Si is lower than its natural abundance level, e.g., lower than about 4.7%, 4%, 3%, 2%, 1%, 0.5% or even 0.1%. Methods for preparing silicon materials (e.g., silicon or silica) with varying levels of silicon isotopes have been developed for the computer industry and are well known in the art, e.g., see Haller, J Applied Physics 77:2857, 1995. In another embodiment, the material includes carbon nuclei with a natural abundance mixture of isotopes 12C (zero-spin, about 98.9%) and 13C (spin-½, about 1.1%). In another embodiment, the level of 13C is higher than its natural abundance level, e.g., higher than about 1.1%, 2%, 5%, 10%, 20%, 30%, 40% or even 50%. In yet another embodiment, the level of 13C is lower than its natural abundance level, e.g., lower than about 1.1%, 1%, 0.8%, 0.6%, 0.4%, 0.2% or even 0.1%. Methods for preparing carbon materials with varying levels of carbon isotopes are also known in the art, e.g., see Graebner et al., Applied Physics Letters, 64:2549, 1994.

In general, the inventive material may include any combination of non-zero spin nuclei and zero-spin nuclei. Taking 29Si and 13C as exemplary non-zero spin nuclei, the invention encompasses imaging agents comprising the following exemplary combinations of nuclei and material: 29Si in a silicon (Si) material (e.g., natural abundance silicon, 29Si enriched silicon or 29Si depleted silicon); 29Si in a silica (SiO₂) material (e.g., natural abundance silica, 29Si enriched silica or 29Si depleted silica): 29Si and/or 13C in a silicon carbide (SiC) material; 13C in a carbon material (e.g., diamond or fullerene); 31P in a silicon (Si) material (e.g., phosphorous doped silicon); 10B or 11B in a silicon (Si) material (e.g., boron doped silicon); etc. In one embodiment, the inventive material includes endohedral fullerenes that incorporate non-zero spin nuclei. For example, an inventive material can include a 15N@60C, 15N@80C, etc. endohedral fullerene (where the 15N@ sign indicates an endohedral fullerene with a core 15N nucleus). 15N is not only a spin-½ nucleus, but it also has a free spin which facilitates the in situ hyperpolarization methods of the present invention. 129Xe and 3He are other exemplary nuclei that can be incorporated within an endohedral fullerene. These endohedral fullerenes can be prepared based on methods in the art, e.g., Fatouros et al., Radiology 240:756, 2006 which describes methods for preparing endohedral metallofullerene particles.

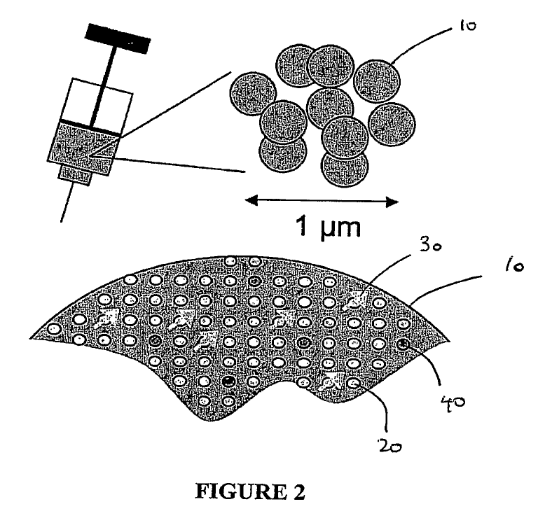

The solid material can be in any form. In certain embodiments, the solid material can be in dry particulate form. For example, the solid material can be in the form of a powder that includes particles with dimensions in the range of 10 nm to 10 μm. In certain embodiments, the particles may have dimensions in the range of 10 nm to 1 μm. In other embodiments, the particles may have dimensions in the range of 10 to 100 nm. It will be appreciated that in certain embodiments, the particles may be combined and compressed for purposes of administration (e.g., in the form of a tablet) and can be formulated along with other ingredients including pharmaceutically acceptable carriers (e.g., binders, lubricants, fillers, etc.). Alternatively, the solid material may be in the form of a suspension with particles having the same range of dimensions (e.g., see FIG. 2). The liquid of the suspension may be aqueous or non-aqueous and may include ingredients that stabilize the suspension (e.g., surfactants) as well as pharmaceutically acceptable carriers. As used herein, the term “pharmaceutically acceptable carrier” means a non-toxic, inert solid, semi-solid or liquid filler, diluting agent, encapsulating material or formulation auxiliary of any type. Remington's Pharmaceutical Sciences Ed. by Gennaro, Mack Publishing, Easton, Pa., 1995 discloses various carriers used in formulating pharmaceutical compositions and known techniques for preparing them. Coloring agents, coating agents, sweetening, flavoring and perfuming agents and preservatives can also be included with an inventive solid material. In general, if a carrier is used, it will be selected based on one or more of the route of administration, the location of the target tissue, the imaging agent being delivered, the time course of delivery of the imaging agent, etc.

In general, the imaging agent may be administered to a subject prior to hyperpolarization using any known route of administration. For example, the imaging agent may be administered orally in the form of a powder, tablet, capsule, suspension, etc. The imaging agent may also be administered by inhalation in the form of a powder or spray. Alternatively, a suspension of the imaging agent may be injected (e.g., intravenously, subcutaneously, intramuscularly, intraperitonealy, etc.) into a tissue or directly into the circulation. Rectal, vaginal, and topical (as by powders, creams, ointments, or drops) administrations are also encompassed.

In certain embodiments, the administered imaging agent is given a sufficient period of time to reach a particular location within the subject prior to in situ hyperpolarization and detection. In one set of embodiments, the imaging agent is present within an internal cavity of the subject at the time of in situ hyperpolarization. This could be a gastrointestinal space (e.g., gut, small intestine, large intestine, etc.) or an airway of the subject. In other embodiments, the imaging agent is present within the circulation of the subject at the time of in situ hyperpolarization. In yet other embodiments, the imaging agent is present within a tissue of the subject at the time of in situ hyperpolarization.

In certain embodiments, the particles of solid material may be modified to include targeting agents that will direct them to a particular cell type (e.g., a tumor cell) or tissue type (e.g., nerve tissue expressing a particular cell-surface receptor). These modified imaging agents will concentrate in regions of the subject that include the cell or tissue type of interest. Proper targeting of these modified imaging agents may require several hours or days post-administration to allow for efficient concentration at the site of interest. Ex vivo hyperpolarization methods with imaging agents that exhibit T1 times on the order of minutes or even hours may be insufficient for such applications. By providing methods for hyperpolarizing imaging agents in situ the present invention enables the imaging of these targeted materials irrespective of their T1 times.

The targeting agents can be associated with particles by covalent or non-covalent bonds (e.g., ligand/receptor type interactions). In one embodiment, patterning of surfaces can be used to promote non-covalent bonds between the targeting agent and inventive particles. Alternatively, a whole host of synthetic methods exist for chemically functionalizing the surfaces of inventive particles to produce surface moieties that form covalent or non-covalent bonds with targeting agents. For example, Bhushan et al., Acta Biomater. 1:327, 2005 describes both chemical conjugation and surface patterning methods for associating biomolecules with silicon particle surfaces. Shirahata et al., Chem. Rec. 5:145, 2005 describes the chemical modification of a silicon surface using monolayers and methods for associating biomolecules with these layers. Nakamura et al., Acc. Chem. Res. 36:807, 2003; Pantarotto et al., Mini Rev. Med. Chem. 4:805, 2004; and Katz et al., Chemphyschem 5:1084, 2004 provide reviews of methods for functionalizing carbon fullerenes and thereby associating them with biomolecules.

It is also to be understood that any ligand/receptor pair with a sufficient stability and specificity may be employed to associate a targeting agent with a particle. In general, the ligand/receptor interaction should be sufficiently stable to prevent premature release of the targeting agent. To give but one example, a targeting agent may be covalently linked with biotin and the particle surface chemically modified with avidin. The strong binding of biotin to avidin then allows for association of the targeting agent and particle. Ahmed et al., Biomed. Microdevices 3:89, 2004 describe this approach for silicon particles. Capaccio et al., Bioconjug. Chem. 16:241, 2005 describe this approach for carbon fullerenes. In general, possible ligand/receptor pairs include antibody/antigen, protein/co-factor and enzyme/substrate pairs. Besides biotin/avidin, these include for example, biotin/streptavidin, FK506/FK506-binding protein (FKBP), rapamycin/FKBP, cyclophilin/cyclosporin and glutathione/glutathione transferase pairs. Other suitable ligand/receptor pairs would be recognized by those skilled in the art.

A variety of suitable targeting agents are known in the art (e.g., see Cotten et al., Methods Enzym. 217:618, 1993; Garnett, Adv. Drug Deliv. Rev. 53:171, 2001). For example, any of a number of different agents which bind to antigens on the surfaces of target cells may be employed. Antibodies to target cell surface antigens will generally exhibit the necessary specificity for the target antigen. In addition to antibodies, suitable immunoreactive fragments may also be employed, such as the Fab, Fab′, or F(ab′)₂ fragments. Many antibody fragments suitable for use in forming the targeting agent are already available in the art. Similarly, ligands for any receptors on the surface of the target cells may suitably be employed as a targeting agent. These include any small molecule or biomolecule (including peptides, lipids and saccharides), natural or synthetic, which binds specifically to a receptor (e.g., a protein or glycoprotein) found at the surface of the desired target cell.

### In Situ Hyperpolarization Methods

Generally, the in situ hyperpolarization methods of the present invention involve providing a subject that contains an inventive imaging agent and hyperpolarizing at least a portion of the non-zero spin nuclei of the agent without removing it from the subject.

In one aspect, the imaging agent includes unpaired electrons. Electron spin transitions in these electrons are excited by radiation that is able to penetrate the subject. In one embodiment, unpaired electrons are provided by doping an inventive imaging agent with either n-type or p-type impurities. The presence of dopants will shorten the T1 time, but only mildly. For example, the T1 times of 29Si in pure silicon doped with various levels of n-type or p-type impurities was investigated in Shulman and Wyluda, Phys. Rev. 103:1127, 1956. The T1 times of 29Si ranged from hours to minutes when the mobile carrier concentration was adjusted from 1×10¹⁴ to 1×10¹⁹. N-type impurities had the greater impact on T1 times. It will be appreciated that any impurity type or level can be used. When selecting a particular level of impurity, the user will need to balance the competition between longer T1 time and ease of hyperpolarization to achieve the appropriate combination of polarization and relaxation. Some applications will favor long T1 times and thus lower impurity levels. Other applications will be less sensitive to T1 and will therefore tolerate higher impurity levels. Precise concentrations of dopants in the inventive solid materials of the invention are readily available commercially (e.g., from Virginia Semiconductor of Fredericksburg, Va.) or can be made using methods known in the semiconductor art (e.g., see Haller, J Applied Physics 77:2857, 1995).

Exemplary and non-limiting materials that can be used as imaging agents in this aspect of the invention include P- or B-doped silicon. In either case, 29Si nuclei can be hyperpolarized and imaged. P-doped silicon provides both unpaired electrons and non-zero spin 31P nuclei (spin-½). In certain embodiments, the 31P nuclei can be hyperpolarized and used for imaging. Boron has two stable isotopes, 10B (spin-3, 20% natural abundance) and 11B (spin- 3/2, 80% natural abundance) which may also be hyperpolarized and imaged. 11B has the advantage of a high NMR receptivity (thus a higher signal for the same polarization density), which may offset the disadvantages of working with a spin higher than ½.

As noted above, the presence of unpaired electrons within the inventive materials of this aspect of the invention will reduce T1 times because of the strong nuclear-electron couplings. As a result, the weaker internuclear couplings (e.g., between 29Si nuclei) will have less of an effect on T1. In such embodiments, the level of zero-spin nuclei in the material may have little impact on T1 times and imaging agents with higher concentrations of non-zero spin nuclei (e.g., 29Si or 13C) may be advantageously used in order to generate maximum signal strength. For example, in a P- or B-doped silicon material, the combined concentration of 28Si and 30Si could be less than 50%, less than 40%, less than 30%, less than 20%, less than 10%, less than 5%, less than 1% or even less than 0.1% of the total concentration of nuclei in the material.

Once the doped imaging agent has been administered to the subject, hyperpolarization is achieved by placing the subject within an applied magnetic field and irradiating the subject with radiation that penetrates the subject and has a frequency that excites electron spin transitions in the unpaired electrons. In certain embodiments, the radiation has a frequency fᵢ within a range of fₑ±fₙ, where fₑ is the Larmor frequency of the unpaired electrons and fₙ is the Larmor frequency of the non-zero spin nuclei. Depending on the exact frequency of the radiation within this range, the linewidth of the ESR (electron spin resonance) spectrum of the unpaired electrons, and the electron-nuclear couplings involved, the electron polarization generated by the radiation will be transferred to the non-zero spin nuclei by one or more of the DNP (dynamic nuclear polarization) mechanisms (i.e., the Overhauser effect, the solid effect and/or thermal mixing).

The hyperpolarized nuclei within the imaging agent can now be detected using appropriate radiation to excite spin transitions of the non-zero spin nuclei. In certain embodiments, this detection step may be performed at a different (e.g., a higher) magnetic field than the hyperpolarization step. In one embodiment, the applied magnetic field may be adjusted in between the two steps. Alternatively, the subject can be physically moved between two fields. Optionally, the nuclear spin signals can also be used to image the spatial distribution of the imaging agent using any known MRI technique, e.g., see MRI in Practice Ed. by Westerbrook et al., Blackwell Publishing, Oxford, UK, 2005. Advantageously, the cycle of in situ hyperpolarization followed by signal acquisition can be repeated for as long as the imaging agent is present within the subject. This allows the imaging agent to be detected and optionally imaged at different points in time.

In one set of embodiments, the subject is an animal, e.g., a mammal. Exemplary mammals include humans, rats, mice, guinea pigs, hamsters, cats, dogs, primates, and rabbits. In one embodiment the subject is a human. The bodies of animals, including the human body, are opaque to radiation with frequencies greater than a certain threshold (fₘₐₓ). For humans, this threshold is about 1 GHz. In order to penetrate animal subjects and thereby excite electron spin transitions within the unpaired electrons of the imaging agent, the radiation will therefore need to have a frequency fᵢ that is less than fₘₐₓ. For humans, this would be less than about 1 GHz, e.g., less than 750 MHz, less than 500 MHz, less than 400 MHz, less than 300 MHz, less than 200 MHz, or even less than 100 MHz. This frequency requirement is independent of the electron effective g-factor in the imaging agent, and translates to a low-field requirement that the applied magnetic field B satisfy B<Bₘₐₓ=hfₘₐₓ/(g μ), where h is Planck's constant, g is the material g-factor, and μₑ is the Bohr magneton. This sets the typical field scale in the millitesla range. For example, an electron resonance frequency of about 300 MHz translates into an applied field of 10 mT. Accordingly, while the radiation frequency f; might range from about 1 GHz to less than 100 MHz for most animal subjects, the applied field B will need to range from about 35 mT to less than about 3 mT. Because this method allows imaging at millitesla applied fields, a significant cost savings may be realized compared to existing tesla-scale MRI systems. In certain embodiments the subject can be imaged within a tesla-scale MRI system after being hyperpolarized at low field.

In another aspect, the in situ hyperpolarization methods of the present invention rely on the in situ creation of unpaired electrons. These methods take advantage of transparent frequency windows that allow optical access to inventive imaging agents that are already within the subject. Most animal subjects including humans have such a transparent window in the near-infrared region for wavelengths ranging from about 600 to 1000 nm or about 1 to 2 eV (e.g., see Vliet et al., J Biomed Opt., 4:392, 1999). Suitable imaging agents absorb energy at wavelengths within this transparent window and produce unpaired electrons as a result of the absorbed energy. For example, if the imaging agent includes silicon, an irradiation wavelength above the band gap of silicon (˜1.1 eV) and below the upper limit of the transparent window (˜2 eV) will penetrate the subject and will be absorbed by the imaging agent to produce unpaired electrons that can be used to hyperpolarize the non-zero spin nuclei of the imaging agent. In certain embodiments, the irradiation wavelength is within the range of about 1.2 to 1.8 eV, or even about 1.4 to 1.6 eV. Any inventive material with these properties may be used in this aspect of the invention. For example, other suitable materials include certain forms of silica that absorb infrared energy including IR-filter silica (e.g., the Schott RG1000 filter from Schott North America, Inc. of Elmsford, N.Y. or the XNite BP2 filter which can be obtained from MaxMax of Carlstadt, N.J.).

In accordance with another embodiment, a hybrid material can be used instead of a material such as silicon that provides both hyperpolarizable nuclei and infrared absorption. Suitable hybrid materials include a first material that absorbs the penetrating infrared energy and a second material with hyperpolarizable nuclei. For example, the first material can be silicon or a suitable silica (e.g., IR-filter silica). The second material has the composition of an inventive imaging agent (i.e., a mixture of zero-spin nuclei and non-zero spin nuclei) and can be selected from any of the aforementioned imaging agents, In general, the first and second materials may be homogeneously or heterogeneously distributed within a hybrid imaging agent. Electrons flow in between the two materials in the presence of penetrating near-infrared radiation. When the radiation is switched off the materials are effectively independent of one another. Because they are not electrically connected together, electrons dissipate. Accordingly, in certain embodiments, the first absorbing material may be physically separate from the second hyperpolarizable material. For example, in one embodiment the first and second materials can be arranged as the shell and core of a particle, respectively. Alternatively the first and second materials can be arranged as a plurality of adjacent layers that could be concentric or parallel.

Once an unpaired electron has been created as a result of radiation within the transparent window, Overhauser excitation at the difference of electron and nuclear resonant frequencies in the range fₑ±fₙ (as described above) may be performed with the electronic states to transfer the polarization of these optically excited electrons to the nuclear states in situ.

The hyperpolarized nuclei within the imaging agent can now be detected using appropriate radiation to excite spin transitions of the non-zero spin nuclei. In certain embodiments, this detection step may be performed at a different (e.g., a higher) magnetic field than the hyperpolarization step. In one embodiment, the applied magnetic field may be adjusted in between the two steps. Alternatively, the subject can be physically moved between two fields. Optionally, the nuclear spin signals can also be used to image the spatial distribution of the imaging agent using any known MRI technique, e.g., see MRI in Practice Ed. by Westerbrook et al., Blackwell Publishing, Oxford, UK, 2005. Advantageously, the cycle of in situ hyperpolarization followed by signal acquisition can be repeated for as long as the imaging agent is present within the subject. This allows the imaging agent to be detected and optionally imaged at different points in time.

### System for Performing In Situ Hyperpolarization

The present invention also provides a novel system for performing in situ hyperpolarization based on the aforementioned near-infrared transparent windows. In general, the system includes (a) a device that is capable of producing an applied magnetic field; (b) a first source of radiation that is capable of penetrating a subject and generating unpaired electrons within an in situ imaging agent; and (c) a second source of radiation for polarizing unpaired electrons at the applied field that have been produced by the first source. In one embodiment, the system includes (a) a device that is capable of producing an applied field in the range of about 1 to 100 mT; (b) a first source of radiation for producing unpaired electrons in an imaging agent which has an energy in the range of about 1 to 2 eV; and (c) a second source of radiation for polarizing the unpaired electrons produced by the first source which has a frequency in the range of about 50 MHz to 3 GHz. In certain embodiments, the device produces an applied field in the range of about 3 to 35 mT, for example about 10 to 25 mT. In certain embodiments, the first source produces radiation with an energy in the range of about 1.2 to 1.8 eV, for example about 1.4 to 1.6 eV. In certain embodiments, the second source produces radiation with a frequency in the range of about 100 MHz to 1 GHz, for example about 300 MHz to about 700 MHz. In one embodiment, the frequency of the second source is tuned to excite electron and/or both electron and nuclear spin transitions at the applied field within the imaging agent and thereby drive dynamic nuclear polarization. Subramanian et al., NMR Biomed. 17:263, 2004 describe OMRI systems that include suitable devices for producing applied fields below 100 mT and methods for coupling these to radiation sources of less than 3 GHz (i.e., the second source of radiation). Here, the inventive system further includes a source of radiation (i.e., the first source of radiation) that is capable of penetrating a subject and producing in situ unpaired electrons within an imaging agent. As previously noted, in one set of embodiments, this source produced radiation with energy in the range of about 1 to 2 eV. A variety of suitable sources are known in the art including a variety of near-infrared sources.

It will be appreciated that the inventive system may include additional components. In particular, the system may include components for detecting the nuclear polarization of the imaging agent. This will typically be in the form of one or more devices (e.g., coils) that have been tuned to the frequency of one or more of the non-zero nuclear spins present within the imaging agent (e.g., 129Xe, 29Si, 31P, 19F, 15N, 13C, 3He, etc.). In one embodiment, the system includes a device for detecting 29Si spin transitions. In another embodiment, the system includes a device for detecting 13C spin transitions. The detection of nuclear polarization may be performed under an applied field in the range of about 1 to 100 mT (i.e., low field detection). Alternatively, the system may include a device that is capable of producing higher fields, e.g., 1 to 10 T and the nuclear polarization may be detected under an applied field in the range of about 1 to 10 T. The inventive system may further include other components that are commonly associated with an MRI machine. For example, the system might include a device for holding a subject at appropriate positions (e.g., within the applied field or fields) and for physically moving the subject into or within the system. The system may also include devices for producing field gradients for imaging purposes. The system may also include a spectrometer for controlling the various components and for processing data signals to and from each component (e.g., to produce images of the imaging agent within the subject).

### Other Embodiments

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

We claim:

1. A method comprising steps of:
providing a subject containing a solid imaging agent that includes non-zero spin nuclei and zero-spin nuclei; and
hyperpolarizing at least a portion of the non-zero spin nuclei without removing the solid imaging agent from the subject.

2. The method of claim 1, wherein the solid imaging agent includes non-zero spin nuclei selected from the group consisting of 129Xe, 29Si, 31P, 19F, 15N, 13C, 11B, and 10B.

3. The method of claim 1, wherein the solid imaging agent includes 29Si nuclei.

4. The method of claim 1, wherein the solid imaging agent includes 13C nuclei.

5. The method of claim 3, wherein the solid imaging agent includes 28Si nuclei.

6. The method of claim 3, wherein the solid imaging agent includes 12C nuclei.

7. The method of claim 4, wherein the solid imaging agent includes 28Si nuclei.

8. The method of claim 4, wherein the solid imaging agent includes 12C nuclei.

9. The method of claim 3, wherein the 29Si nuclei are present at natural abundance levels.

10. The method of claim 3, wherein the 29Si nuclei are present at lower than natural abundance levels.

11. The method of claim 3, wherein the 29Si nuclei are present at higher than natural abundance levels.

12. The method of claim 1, wherein the solid imaging agent includes 29Si nuclei in a silicon material.

13. The method of claim 1, wherein the solid imaging agent includes 29Si nuclei in a silica material.

14. The method of claim 1, wherein the solid imaging agent includes 29Si and/or 13C nuclei in a silicon carbide material.

15. The method of claim 1, wherein the solid imaging agent includes 13C nuclei in a carbon material.

16. The method of claim 1, wherein the solid imaging agent includes 31P nuclei in a silicon material.

17. The method of claim 1, wherein the solid imaging agent includes 10B and/or 11B nuclei in a silicon material.

18. The method of claim 1, wherein the solid imaging agent includes 15N nuclei in a carbon material.

19. The method of claim 18, wherein the carbon material is an endohedral fullerene.

20. The method of claim 1, wherein the T1 time of the non-zero spin nuclei is greater than one hour.

21. The method of claim 1, wherein the solid imaging agent was administered to the subject in the form of particles.

22. The method of claim 21, wherein the particles have dimensions in the range of 10 nm to 10 μm.

23. The method of claim 21, wherein the particles have dimensions in the range of 10 nm to 1 μm.

24. The method of claim 21, wherein the particles have dimensions in the range of 10 nm to 100 nm.

25. The method of claim 1, wherein the solid imaging agent was administered to the subject in the form of a suspension of particles.

26. The method of claim 1, wherein the subject is an animal.

27. The method of claim 1, wherein the subject is a mammal.

28. The method of claim 1, wherein the subject is selected from the group consisting of rats, mice, guinea pigs, hamsters, cats, dogs, primates and rabbits.

29. The method of claim 1, wherein the subject is a human.

30. The method of claim 1, wherein the step of providing comprises a step of:
administering the solid imaging agent to the subject.

31. The method of claim 30, wherein the solid imaging agent is administered orally.

32. The method of claim 30, wherein the solid imaging agent is administered by inhalation.

33. The method of claim 30, wherein the solid imaging agent is administered by injection.

34. The method of claim 30, wherein the step of providing further comprises a step of:
waiting for a sufficient period of time to allow the solid imaging agent to reach a particular location within the subject before performing the step of hyperpolarizing.

35. The method of claim 34, wherein the solid imaging agent is present within an internal cavity of the subject at the time of hyperpolarization.

36. The method of claim 34, wherein the solid imaging agent is present within a gastrointestinal space of the subject at the time of hyperpolarization.

37. The method of claim 34, wherein the solid imaging agent is present within an airway of the subject at the time of hyperpolarization.

38. The method of claim 34, wherein the solid imaging agent is present within a circulatory system of the subject at the time of hyperpolarization.

39. The method of claim 34, wherein the solid imaging agent is present within a tissue of the subject at the time of hyperpolarization.

40. The method of claim 1 further comprising a step of:
detecting the hyperpolarized non-zero spin nuclei while the solid imaging agent is present within the subject.

41. The method of claim 40, wherein the spatial distribution of the solid imaging agent within the subject is imaged by magnetic resonance imaging.

42. The method of claim 1, wherein the steps of hyperpolarizing and detecting are repeated at least once without removing the solid imaging agent from the subject.

43. The method of claim 42, wherein the spatial distribution of the solid imaging agent within the subject is monitored over time.

44. The method of claim 1, wherein the steps of hyperpolarizing and detecting are performed at the same magnetic field.

45. The method of claim 1, wherein the steps of hyperpolarizing and detecting are performed at different magnetic fields.

46. The method of claim 45, wherein the organism is physically moved between two different magnetic fields.

47. The method of claim 45, wherein the steps of hyperpolarizing and detecting are performed using an adjustable magnetic field source.

48. The method of claim 1, wherein the solid imaging agent is associated with a targeting agent that binds with an antigen present on the surface of a cell.

49. The method of claim 48, wherein the targeting agent is an antibody or an immunoreactive fragment of an antibody for the antigen present on the surface of the cell.

50. The method of claim 48, wherein the targeting agent is a ligand and the antigen present on the surface of the cell is a receptor for the ligand.

51. The method of claim 1, wherein the solid imaging agent includes unpaired electrons and the step of hyperpolarizing comprises steps of:
placing the subject within an applied magnetic field; and
irradiating the subject with radiation that penetrates the subject and excites electron spin transitions in the unpaired electrons.

52. The method of claim 51, wherein the radiation has a frequency fᵢ in the range of fₑ±fₙ, where fₑ is the Larmor frequency of the unpaired electrons and fₙ is the Larmor frequency of the non-zero spin nuclei.

53. The method of claim 51, wherein the solid imaging agent is doped with an n-type impurity.

54. The method of claim 51, wherein the solid imaging agent is doped with a p-type impurity.

55. The method of claim 51, wherein the solid imaging agent comprises silicon doped with phosphorous.

56. The method of claim 51, wherein the solid imaging agent comprises silicon doped with boron.

57. The method of claim 51, wherein the radiation has a frequency that is lower than about 1 GHz and the applied magnetic field is lower than about 35 mT.

58. The method of claim 51, wherein the radiation has a frequency in the range of about 100 to 750 MHz.

59. The method of claim 51, wherein the applied magnetic field is in the range of about 3 to 25 mT.

60. The method of claim 51, wherein the subject is opaque to radiation with a frequency greater than 1 GHz.

61. The method of claim 1, wherein the step of hyperpolarizing comprises steps of:
placing the subject within an applied magnetic field; and
irradiating the subject with a first form of radiation that penetrates the subject, wherein the energy of the first form of radiation and the composition of the solid imaging agent are such that the first form of radiation produces unpaired electrons within the solid imaging agent.

62. The method of claim 61, wherein the first form of radiation has an energy in the range of about 1 to 2 eV.

63. The method of claim 61, wherein the first form of radiation has an energy in the range of about 1.2 to 1.8 eV.

64. The method of claim 61, wherein the first form of radiation has an energy in the range of about 1.4 to 1.6 eV.

65. The method of claim 61, wherein the solid imaging agent comprises silicon.

66. The method of claim 65, wherein the first form of radiation has an energy that is greater than about 1.2 eV.

67. The method of claim 61, wherein the solid imaging agent comprises silica.

68. The method of claim 61, wherein the step of hyperpolarizing further comprises a step of:
irradiating the subject with a second form of radiation that penetrates the subject and excites electron spin transitions in the unpaired electrons.

69. The method of claim 68, wherein the second form of radiation has a frequency that is lower than about 1 GHz and the applied magnetic field is lower than about 35 mT.

70. The method of claim 68, wherein the second form of radiation has a frequency in the range of about 100 to 750 MHz.

71. The method of claim 68, wherein the applied magnetic field is in the range of about 3 to 25 mT.

72. The method of claim 61, wherein the solid imaging agent is a hybrid material that includes a first material that absorbs the first form of radiation to produce unpaired electrons and a second material that includes non-zero spin nuclei and zero-spin nuclei.

73. The method of claim 72, wherein the first material includes silicon.

74. The method of claim 72, wherein the first material includes silicon doped with an n-type impurity.

75. The method of claim 72, wherein the first material includes silicon doped with a p-type impurity.

76. The method of claim 72, wherein the first material includes silicon doped with phosphorous.

77. The method of claim 72, wherein the first material includes silicon doped with boron.

78. The method of claim 72, wherein the first material includes silica.

79. The method of claim 72, wherein the first and second materials are homogeneously distributed within the solid imaging agent.

80. The method of claim 72, wherein the first and second materials are heterogeneously distributed within the solid imaging agent.

81. The method of claim 72, wherein the first material forms a shell surrounding a core of the second material.

82. The method of claim 72, wherein the first and second materials are arranged as adjacent layers.

83. The method of claim 72, wherein the T1 time of the non-zero spin nuclei of the second material is greater than one hour.

84. A system for hyperpolarizing a solid imaging agent while present in a subject comprising:
a device capable of producing a magnetic field;
a first source of radiation that is capable of penetrating a subject and generating unpaired electrons within the solid imaging agent; and
a second source of radiation for polarizing unpaired electrons at the applied field that have been produced by the first source of radiation.

85. The system of claim 84, wherein the device produces an applied field in the range of about 1 to 100 mT; the first source of radiation has an energy in the range of about 1 to 2 eV; and the second source of radiation has a frequency in the range of about 50 MHz to 3 GHz.

86. The system of claim 85, wherein the device produces an applied field in the range of about 3 to 35 mT.

87. The system of claim 85, wherein the device produces an applied field in the range of about 10 to 25 mT.

88. The system of claim 85, wherein the first source of radiation has an energy in the range of about 1.2 to 1.8 eV.

89. The system of claim 85, wherein the first source of radiation has an energy in the range of about 1.4 to 1.6 eV.

90. The system of claim 85, wherein the second source of radiation has a frequency in the range of about 100 MHz to 1 GHz.

91. The system of claim 85, wherein the second source of radiation has a frequency in the range of about 300 MHz to about 700 MHz.
